(19) United States
(12) Patent Application Publication
(10) Pub. No.: US 2017/0059671 A1
(43) Pub. Date: Mar. 2, 2017

(54) MEDICAL IMAGING APPARATUS WITH A HOUSING

(71) Applicant: Siemens Healthcare GmbH, Erlangen (DE)

(72) Inventor: Bernd Maciejewski, Markt Erlbach (DE)

(73) Assignee: Siemens Healthcare GmbH, Erlangen (DE)

(21) Appl. No.: 15/248,319

(22) Filed: Aug. 26, 2016

(30) Foreign Application Priority Data
Aug. 27, 2015 (DE) 102015216409.8

(51) Int. Cl. G01R 33/28; A61B 5/055

(52) U.S. Cl. CPC G01R 33/28; A61B 5/055

(57) ABSTRACT

A medical imaging apparatus has a scanner and a housing for the scanner. The scanner housing has at least one housing shell that has at least one first layer and at least one second layer. The at least one first layer has at least one stiffening element and the at least one second layer has a layer element with at least a portion thereof that is translucent.

### BACKGROUND OF THE INVENTION

### Field of the Invention

The present invention concerns a medical imaging apparatus with a scanner and a housing having at least one housing shell.

### Description of the Prior Art

Medical imaging apparatuses frequently require a number of supply lines. For example, a magnetic resonance apparatus requires data cables, energy lines and/or energy cables, water lines, quench pipes, etc. Such supply lines are frequently passed from a ceiling to the medical imaging apparatus.

Conventionally a housing has been used to encase such supply lines. It is often not possible to arrange such a housing on the ceiling, because the ceilings are frequently suspended and the support structure required for the housing is therefore not present. Also, it must be possible to configure the housing in a flexible manner, because the heights between the medical imaging apparatus and the ceiling vary depending on the installation site or the examination space.

### SUMMARY OF THE INVENTION

An object of the present invention is to provide a housing that is easy to mount and has a high level of rigidity to encase supply lines for a medical imaging apparatus.

The invention is based on a medical imaging apparatus with a scanner and a housing, the housing having at least one housing shell.

In accordance with the invention, the at least one housing shell has at least one first layer and at least one second layer, the at least one first layer having at least one stiffening element and the at least one second layer having an at least partially translucent layer element.

The at least one stiffening element here gives the at least one housing shell the required rigidity, allowing the housing shell to achieve a high level of strength and ease of mounting. The at least one housing shell has a shape based on the shape of the at least one stiffening element. The translucent layer element allows the housing shell to achieve a beneficial visual configuration, with which light effects are visible to the observer. It is possible thus to establish a degree of translucence on the at least one housing shell as a function of the layer thickness of the at least one second layer, in particular of the translucent layer element.

In this context a translucent layer element refers to a layer element with translucent properties.

It is further proposed for the medical imaging apparatus to have supply lines leading to the scanner, and the at least one housing shell has a casing that at least partially encloses the supply lines. This allows a housing shell to be provided that is particularly easy to mount and has a high level of rigidity to encase the supply lines.

In a further embodiment of the invention, the at least one second layer has a layer element that is transparent. This allows the at least one first layer, in particular stiffening elements, and/or components that are protected and/or enclosed by the housing, to be advantageously visible. The translucent layer element and the transparent layer element can be configured as a single piece with one another.

The at least one second layer preferably has an elastic layer element. The elastic layer element and the translucent layer element are preferably configured as a single piece with one another. Elasticity of this layer element of the at least one second layer allows a housing to be provided that can be configured in a particularly flexible manner. The elastic layer element also achieves sound insulation for the housing during a medical imaging examination. This advantage is particularly beneficial for a magnetic resonance apparatus, because the scanner thereof generates an undesirably high level of noise during the magnetic resonance examination.

In a further embodiment of the invention, the translucent layer element has a foam material and/or a casting compound material. This allows the production process for producing the at least one housing shell to be particularly simple and economical. It is also possible to provide a particularly light housing shell that, because of its low weight, allows simple mounting. Such a casting compound material can be, for example, a casting resin, in particular a polyester resin and/or an epoxy resin and/or a silicone resin and/or a vinyl resin and/or polyurethane resin, etc. The material of the casting compound may also be selected so as to satisfy safety criteria, such as UL 94 safety criteria for impeding the flammability of plastics. The casting compound can be or include a casting resin that satisfies such criteria.

In a further embodiment, the at least one stiffening element is formed as a rigid foam. This provides a particularly light and economical stiffening element for the at least one housing shell. The at least one stiffening element preferably is a thermoplastic and therefore thermally formable rigid foam.

In another embodiment, the at least one first layer has two or more stiffening elements, which are arranged at a distance from one another in the direction of the layer length. This allows a gap between the stiffening elements within the at least one housing shell and the at least one housing shell thus can be collapsed and/or folded for transportation. This allows easy transportation of the at least one housing shell.

In another embodiment of the invention, the at least one first layer has two or more stiffening elements and the two or more stiffening elements are arranged within a grid structure of the at least one first layer. This allows the shape of the at least one first layer of the at least one housing shell to be configured in a particularly flexible manner.

In a further embodiment of the invention, the at least one stiffening element of the at least one first layer is arranged, after being pre-shaped, within the at least one housing shell. This allows simple and time-saving mounting, as the at least one housing shell already assumes an essentially final shape due to the at least one pre-shaped stiffening element, and time-consuming shaping of the at least one housing shell during a mounting operation can advantageously be avoided.

In a further embodiment, the at least one stiffening element of the at least one first layer has two or more stiffening regions, which are connected to one another on a first surface of the first layer. This embodiment of the invention allows a high level of flexibility of the at least one housing shell. It allows the at least one housing shell to be matched to any housing shapes in a simple manner, even during an operation to mount the at least one housing shell.

In a further embodiment of the invention, the at least one housing shell has a first layer and two second layers, the one first layer being arranged between the two second layers. This allows advantageous sound insulation and a high level of flexibility of the at least one housing shell due to the two second layers. A high level of rigidity and therefore advantageous and simple mounting can also be achieved due to the one first layer. A housing shell with such a structure can also be matched particularly easily to any shape for a component housing.

Alternatively, the at least one housing shell can have two first layers and one second layer, the one second layer being arranged between the two first layers. This type of arrangement of the individual layers within the at least one housing shell allows a compact structure to be achieved with a thin overall shell thickness, of a maximum of 10 mm, particularly advantageously maximum 8 mm and particularly preferably between 6 mm and 7 mm. Advantageous sound insulation and a high level of flexibility of the at least one housing shell can also be achieved due to the one second layer in conjunction with a high level of rigidity due to the two first layers. A housing shell with such a structure can also be matched easily to any shape of component casing.

In a further embodiment of the invention, at least one of the two first layers has a coating layer on an outwardly facing surface of the at least one housing shell. This allows advantageous protection of the at least one housing shell. It also allows the at least one housing shell to be highly durable.

The coating layer can be a fire protection coating, thereby improving the safety of the at least one housing shell and also the entire medical imaging apparatus. This also allows safety criteria for operation of the medical imaging apparatus to be met so there is no need for additional safety measures with respect to fire protection when mounting the medical imaging apparatus. The coating layer can have multiple layers, and an outer layer of the coating layer can be a flexible protective coating and an inner layer of the coating layer can be the fire protection coating. The inner layer and/or the fire protection coating can be designed as elastic.

In another embodiment of the invention, the at least one housing shell has a fastening unit. The fastening unit is arranged on at least one end region of the housing shell. This achieves a space-saving arrangement of the fastening unit. The fastening unit preferably is a latching element and/or a form-fit element and/or any further type of fastening element that appear expedient to those skilled in the art.

An arrangement of the fastening unit on the at least one housing shell that is economical with respect to components and cost is achieved when the fastening unit is cast with the at least one first layer and/or with the at least one second layer. This also allows a high level of stability of fastening to be achieved between the fastening unit and the at least one first layer and/or the at least one second layer.

In a further embodiment of the invention, the at least one housing shell has an overall thickness and the overall thickness is maximum 15 mm. The overall thickness is preferably a maximum of 12 mm and more preferably a maximum of 9 mm to 10 mm.

This provides a thin and space-saving design of the at least one housing shell for the housing casing for incoming lines and/or supply lines for the medical imaging apparatus.

### BRIEF DESCRIPTION OF THE DRAWINGS

FIG. 1 schematically illustrates a medical imaging apparatus with a housing in accordance with the invention.

FIG. 2 schematically illustrates a first exemplary embodiment of a housing shell of the housing in accordance with the invention.

FIG. 3 schematically illustrates a first embodiment of a first layer of the housing shell in accordance with the invention.

FIG. 4 schematically illustrates a second embodiment of the first layer of the housing shell in accordance with the invention.

FIG. 5 schematically illustrates a second exemplary embodiment of a housing shell of the housing in accordance with the invention.

### DESCRIPTION OF THE PREFERRED EMBODIMENTS

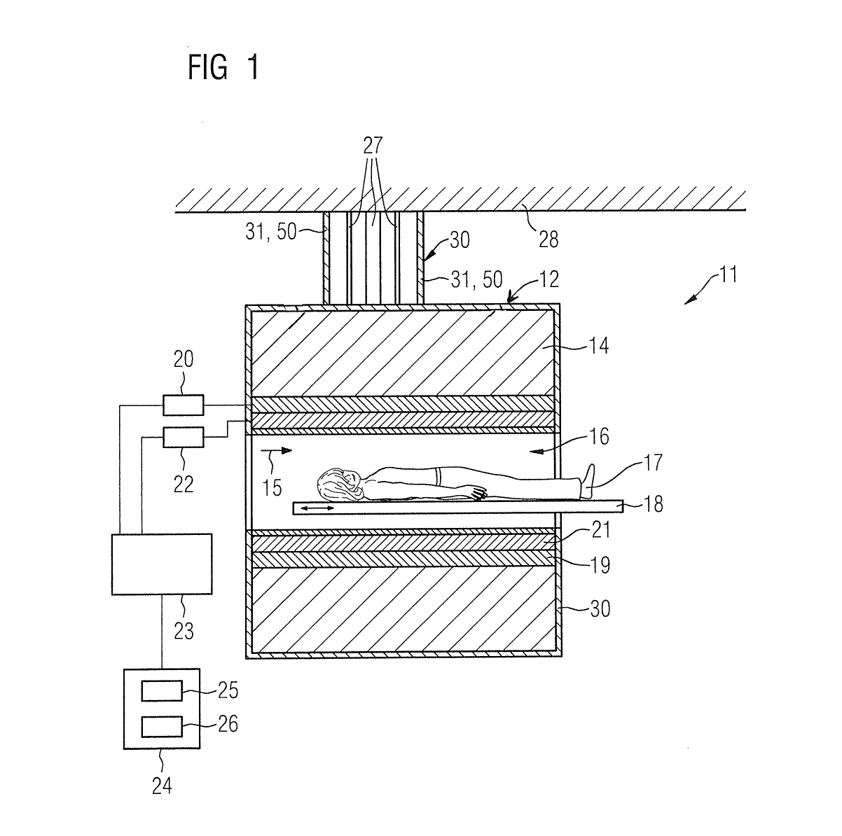

FIG. 1 schematically shows a medical imaging apparatus. In the exemplary embodiment the medical imaging apparatus is formed by a magnetic resonance apparatus 11, the present invention being described as an example with reference to the magnetic resonance apparatus 11. The present invention is not restricted to the configuration of the medical imaging apparatus as a magnetic resonance apparatus 11 and further embodiments of the medical imaging apparatus are conceivable, such as a PET apparatus (Positron Emission Tomography apparatus), a computed tomography apparatus, etc.

The magnetic resonance apparatus 11 has a data acquisition scanner 12. The scanner 12 has a superconducting basic field magnet 14 that generates a strong and constant basic magnetic field 15. The scanner 12 has a patient receiving region 16 for receiving a patient 17. In the exemplary embodiment the patient receiving region 16 is configured as cylindrical and it is enclosed in a cylindrical manner in a circumferential direction by the scanner 12. A different configuration of the patient receiving region 16 is conceivable. The patient 17 can be moved into the patient receiving region 16 by a patient support 18 of the magnetic resonance apparatus 11.

The scanner 12 also has a gradient coil arrangement 19 that generates magnetic field gradients, which are used for spatial encoding during imaging. The gradient coil arrangement 19 is controlled by a gradient control processor 20 of the magnetic resonance apparatus 11. The scanner 12 also has a radio-frequency antenna that is controlled by a radio-frequency antenna control processor 22 so as to radiate radio frequency magnetic resonance sequences into an examination space, which is essentially formed by the patient receiving region 16 of the scanner 12. The radio-frequency sequences (pulses) excite certain nuclear spins in the patient 15 so as to have a magnetization that deviates from the field lines of the basic magnetic field 15. As the excited nuclear spins relax, they emit magnetic resonance signals.

The magnetic resonance apparatus 11 has a system control computer 23 that controls the basic field magnet 14, the gradient control processor 20 and to control the radio-frequency antenna control processor 22. The system control computer 23 controls the magnetic resonance apparatus 11 centrally, for example to perform a predetermined gradient echo sequence for imaging. The system control computer 23 also has an analysis unit (not shown in detail) for analyzing medical image data acquired during the magnetic resonance examination. The magnetic resonance apparatus 11 also has a user interface 24, which is connected to the system control computer 23. Control information, such as imaging parameters, as well as reconstructed magnetic resonance images can be displayed to medical operators on a display unit 25, for example on at least one monitor, of the user interface 24. The user interface 24 also has an input unit 26, which medical operators can use during a data acquisition procedure to input information and/or parameters.

The magnetic resonance apparatus 11 has a housing 30, which at least partially encloses the scanner 13. The housing 30 has at least one housing shell 31 that encases supply lines 27 proceeding to the magnetic resonance apparatus 11. The supply lines 27 can be, for example, data cables, energy lines and/or energy cables, water lines, quench pipes, etc. Such supply lines 27 proceed from the ceiling 28 of an examination space, in which the magnetic resonance apparatus 11 is situated, to the magnetic resonance apparatus 11. The housing 30 has two or more housing shells 31, 50 to encase the supply lines 27.

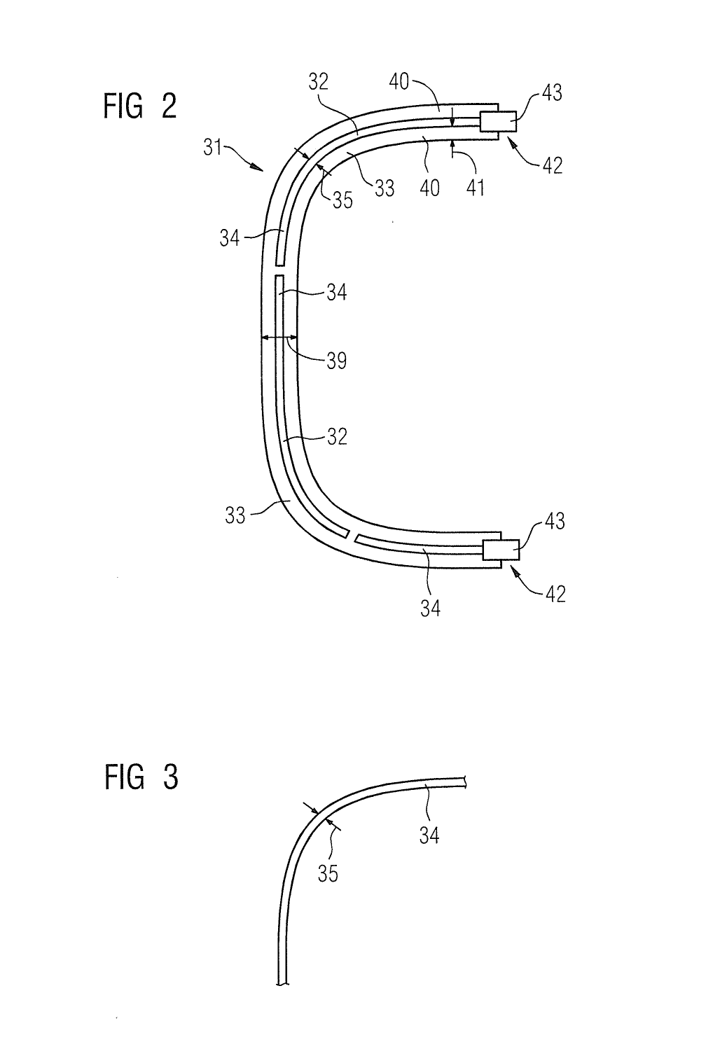

One embodiment of the two or more housing shells 31 of the housing 30 is shown as an example in FIG. 2. The exemplary embodiment of the housing shell 31 shown in FIG. 2 is enclosed by the housing 30 so as to encase the supply lines 27. The housing shell 31 has a first layer 32 and two second layers 33.

In the exemplary embodiment shown in FIG. 2, the first layer 32 has two stiffening elements 34. In a different embodiment of the first layer 32, it also has just one stiffening element 34 or even more than two stiffening elements 34. In the exemplary embodiment the individual stiffening elements 34 are formed as a rigid foam, in particular a thermoplastic rigid foam, so that the stiffening elements 34 are light but stable within the housing shell 31. The individual stiffening elements composed of thermoplastic rigid foam can be shaped particularly simply as required by thermoforming and therefore can be matched easily to a desired geometry. The first layer 32, in particular the individual stiffening elements 34 of the first layer 32, have a maximum layer thickness 35 of 8 mm, preferably a maximum 5 mm and most maximum 2 mm to 4 mm.

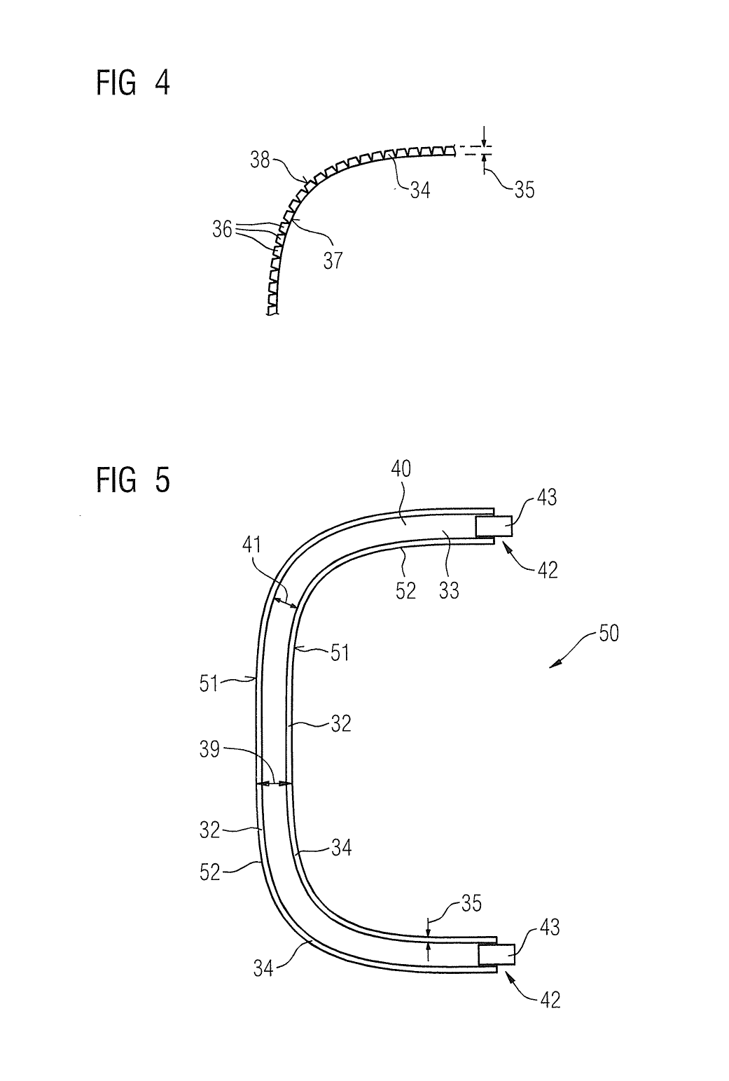

Different exemplary embodiments of the stiffening elements 34 are shown as examples based on a single stiffening element 34 in FIGS. 3 and 4. FIG. 3 shows a first exemplary embodiment of the stiffening element 34, in which the stiffening element 34 is pre-shaped. It is thus possible to use the stiffening element 34, in particular the shape of the stiffening element 34, to determine the shape of the housing shell 31. The stiffening element 34 is preferably thermally pre-shaped.

FIG. 4 shows a second exemplary embodiment of the stiffening element 34. The stiffening element 34 here has a number of stiffening regions 36, which are connected to one another on a first surface 37 of the first layer 32, in particular of the stiffening element 34. On a second surface 38 of the first layer 32, in particular of the stiffening element 34, the individual stiffening regions 36 are configured separately from one another. The second surface 38 of the first layer 32 here is opposite the first surface 37 of the first layer 32. This allows particularly flexible shaping of the housing shell 31, which can also compensate for tolerances of the housing 30 and/or also tolerances in relation to the housing 30.

The two stiffening elements 34 within the first layer 32 of the housing shell 31 in

FIG. 2 are arranged at a distance from one another. The space between the two stiffening elements 34 allows the housing shell 31 to be folded, which is advantageous for transportation of the housing shell 31.

The first layer 32 is arranged in a center region within the housing shell 31. The first layer 32 is arranged between the two second layers 33 (FIG. 2) in the direction of the overall thickness 39 of the housing shell 31.

The two second layers 33 are configured in the same manner and each have a translucent layer element 40. The translucent layer element 40 can also be configured so that at least a portion thereof is transparent. The two second layer elements 40 are also configured as elastic and include a foam material and/or a casting compound. The two second layers 33, in particular the individual layer elements 40 of the second layers 33, have a maximum layer thickness 41 of 5 mm, preferably a maximum of 2 mm to 4 mm.

To produce the housing shell 31, the first layer 32, in particular the stiffening elements 34, is preferably moved into position within a mold and the foam material and/or the casting compound of the layer elements 40 of the second layers 33 is cast around it. The stiffening elements 34 of the first layer 32 are thus cast between the two second layers 33, in particular between the two elastic, translucent layer elements 40.

The casting compound can be, for example, a casting resin, in particular a polyester resin and/or an epoxy resin and/or a silicone resin and/or a vinyl resin and/or polyurethane resin, etc. The casting compound may also satisfy particular safety criteria, such as UL 94 safety criteria for impeding the flammability of plastics, so the casting compound includes a casting resin with that property.

The housing shell 31 also has a fastening unit 42 to fasten the housing shell 31 to a further housing shell 31 and/or further components. In the exemplary embodiment, the fastening unit 42 is formed by two latching elements 43 on the housing shell 31. In an alternative embodiment of the fastening unit 42, it is a form-fit element and/or a force-fit element and/or further types of fastening elements that appear expedient to those skilled in the art. In one alternative embodiment of the housing shell 31 and/or of the fastening unit 42, the fastening unit 42 can have more than two fastening elements and/or latching elements 43, for example four fastening elements and/or latching elements 43.

The two latching elements 43 of the fastening unit 42 are each arranged on an end region of the housing shell 31. The two end regions here are arranged opposite one another on the housing shell 31 in the direction of a longitudinal extension of the housing shell 31. To fasten and/or arrange the two latching elements 43 on the first layer 32, such as on the stiffening element 34, and/or on at least one of the two second layers 33, such as the elastic and translucent layer element 40, the two latching elements 43 are each preferably cast together with the first layer 32 and/or at least one of the two second layers 33.

The layered overall structure of the housing shell 31 is particularly thin and light. The overall thickness 39 of the housing shell 31 is a maximum of 15 mm, preferably 12 mm and most preferably 9 mm to 10 mm.

FIG. 5 shows an alternative exemplary embodiment of a housing shell 50 of the housing 30. Essentially identical components, features and functions are shown in principle with identical reference characters. The description which follows is essentially restricted to the differences compared with the exemplary embodiment in

FIGS. 2 to 4, reference being made to the description of the exemplary embodiment in FIGS. 2 to 4 for identical components, features and functions.

The further embodiment of two or more housing shells 50 of the housing 30 is illustrated as an example based on a housing shell 50 in FIG. 5. The housing shell 50 has two first layers 32 and one second layer 33. The one second layer 33 here is arranged between the two first layers 32. The two first layers 32, in particular the individual stiffening elements 34 of the two first layers 32, each have a maximum layer thickness 35 of 3 mm, preferably a maximum of 2 mm and most preferably a maximum of 1.0 mm to 1.5 mm.

A material property of the two first layers 32 here corresponds to a material property of the first layer 32 in the exemplary embodiment in FIGS. 2 to 4. Similarly a material property of the one second layer 33 also corresponds to a material property of the two second layers 33 in the exemplary embodiment in FIGS. 2 to 4. The second layer 33, in particular the elastic and translucent layer element 40 of the second layer 33, has a maximum layer thickness 41 of 7 mm, preferably a maximum of 4 mm to 5 mm.

Each of the two first layers 32 has a surface 51 of the housing shell 50, the two surfaces 51 of the housing shell 50 being arranged on opposing sides in the direction of an overall thickness 39 of the housing shell 50.

The two first layers 32, in particular the stiffening elements 34, have a coating layer 52 in each case on the outward facing surface 51 to protect the housing shell 50. The coating layer 52 preferably is a flexible fire protection coating. The coating layer 52 may be composed of a number of layers, with an outer layer of the coating layer 52 being a flexible protective coating and an inner layer of the coating layer 52 being the flexible fire protection coating.

To produce the housing shell 50, the first layers 32, in particular the stiffening elements 34, are moved into position within a mold and the foam material and/or the casting compound of the layer elements 40 of the second layers 33 is cast into a region between the two first layers 32. The casting compound can similarly be a casting resin, in particular a polyester resin and/or an epoxy resin and/or a silicone resin and/or a vinyl resin and/or polyurethane resin, etc. The casting compound may also satisfy particular safety criteria, such as UL 94 safety criteria for impeding the flammability of plastics, and the casting compound has a casting resin with this property.

The housing shell 50 also has a fastening unit 42, which is configured as in the description relating to FIG. 2. The fastening unit 50 is also arranged on the housing shell 42 as in the description relating to FIG. 2.

The layered overall structure of the housing shell 50 in FIG. 5 is particularly thin and light. The overall thickness 39 of the housing shell 50 is a maximum of 12 mm, preferably 9 mm and most preferably 6 mm to 7 mm.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical imaging apparatus comprising:
a data acquisition scanner;
a scanner housing that at least partially encloses said scanner;
said scanner housing comprising at least one housing shell; and
said at least one housing shell comprising at least one first layer and at least one second layer, said at least one first layer comprising at least one stiffening element and said at least one second layer having at least one portion thereof formed as a translucent layer element.

2. A medical imaging apparatus as claimed in claim 1 comprising a plurality of supply lines proceeding to said scanner, and wherein said at least one housing shell forms a casing that at least partially encases said supply lines.

3. A medical imaging apparatus as claimed in claim 1 wherein said at least one second layer comprises at least a portion thereof that is a transparent layer element.

4. A medical imaging apparatus as claimed in claim 1 wherein said at least one second layer comprises at least a portion thereof formed by an elastic layer element.

5. A medical imaging apparatus as claimed in claim 1 wherein said translucent layer element comprises at least one material selected from the group consisting of foam material and casting compound material.

6. A medical imaging apparatus as claimed in claim 1 wherein said at least one stiffening element is comprised of rigid foam.

7. A medical imaging apparatus as claimed in claim 1 wherein said at least one first layer comprises a plurality of stiffening elements respectively situated at respective distances from each other along a layer length of said at least one first layer.

8. A medical imaging apparatus as claimed in claim 1 wherein said at least one stiffening element of said at least one first layer is arranged in said at least one first layer after being pre-shaped within said at least one housing shell.

9. A medical imaging apparatus as claimed in claim 1 wherein said at least one stiffening element of said at least one first layer comprises multiple stiffening regions that are connected to each other on a first surface of said at least one first layer.

10. A medical imaging apparatus as claimed in claim 1 wherein said at least one housing shell comprises one first layer and two second layers, said one first layer being situated between said two second layers.

11. A medical imaging apparatus as claimed in claim 1 wherein said at least one housing shell comprises two first layers and one second layer, said one second layer being situated between said two first layers.

12. A medical imaging apparatus as claimed in claim 11 wherein at least one of said two first layers comprises a coating layer on an outwardly facing surface of said at least one housing shell.

13. A medical imaging apparatus as claimed in claim 12 wherein said coating layer is a fire protection coating.

14. A medical imaging apparatus as claimed in claim 1 wherein said at least one housing shell comprises a fastening unit arranged on at least one end region of said housing shell.

15. A medical imaging apparatus as claimed in claim 14 wherein said fastening unit is cast together with said at least one first layer.

16. A medical imaging apparatus as claimed in claim 14 wherein said fastening unit is cast together with said at least one second layer.

17. A medical imaging apparatus as claimed in claim 14 wherein said fastening unit is cast together with said at least one first layer and said at least one second layer.

18. A medical imaging apparatus as claimed in claim 1 wherein said at least one housing shell has a total thickness, and wherein said total thickness is a maximum of 15 mm.
